(19) United States
(12) Patent Application Publication
(10) Pub. No.: US 2023/0398351 A1
(43) Pub. Date: Dec. 14, 2023

(54) METHODS FOR TREATING HORMONAL DISORDERS

(71) Applicant: Novocure GmbH, Root D4 (CH)

(72) Inventors: Lilach AVIGDOR, Haifa (IL); Tali VOLOSHIN-SELA, Haifa (IL)

(73) Assignee: Novocure GmbH, Root D4 (CH)

(21) Appl. No.: 18/209,810

(22) Filed: Jun. 14, 2023

Related U.S. Application Data
(60) Provisional application No. 63/352,160, filed on Jun. 14, 2022.

(51) Int. Cl. A61N 1/36; A61K 45/06

(52) U.S. Cl. CPC A61N 1/36007; A61K 45/06; A61N 1/36171

(57) ABSTRACT

A method of treating a subject, the method comprises applying an alternating electric field focused on a liver of the subject at a frequency between approximately 50 kHz and approximately 10,000 kHz, where the subject has hormonal disorder and/or polycystic ovary syndrome.

### CROSS-REFERENCE TO RELATED APPLICATIONS

This application claims priority to U.S. Provisional Application 63/352,160 filed on Jun. 14, 2022, the entire contents of which are expressly incorporated herein by reference.

### BACKGROUND

Alternating electric fields are low intensity alternating electric fields within the intermediate frequency range, which may be used to treat certain disorders as described in U.S. Pat. No. 7,565,205. For example, alternating electric fields are induced non-invasively into a region of interest by transducers placed directly on the subject's body and applying AC voltages between the transducers. AC voltage is applied between a first pair of transducers for a first interval of time to generate an electric field with field lines generally running in the front-back direction. Then, AC voltage is applied at the same frequency between a second pair of transducers for a second interval of time to generate an electric field with field lines generally running in the right-left direction. The system then repeats this two-step sequence throughout the treatment.

Polycystic ovary syndrome (PCOS) is a complex and common endorinopathy that affects 5-7% of reproductive age women world-wide. The clinical manifestation of PCOS varies from a mild menstrual disorder to severe disturbance of reproductive and metabolic disfunctions. The pathogenesis of PCOS is largely unknown, however, metabolic disorder, such as insulin reisistance, is common among PCOS pateients. Compensatory hyperinsulinemia can directly stimulate ovarian and adrenal secreation of androgen and decrease sex hormone-binding globulin (SHBG) synthesis, resulting in an creased bioavailability of free testosterone level. As such, SHBG is found to be an early biomarker and therapeutic target in PCOS. SHBG is producted primarily by hepatocytes and is also expressed in the brain, uterus, breast, ovary, and others. It binds and transports testosterone, estradiol and other sex steroids in the plasma, reduces their metabolism clearance rate and affects their bioavailablity. The level of circulating testosterone is largely influenced by SHBG as about 65% of testosterone is bound to SHBG.

PCOS has a significant effect on hyperandrogenemia, cancer, insulin resistance, hypercholesterolemia, hypertension, obesity, sleep amenorrhea, and cardiovascular disease. There are certain options, such as bariaric surgery, laparoscopic ovarian diathermy, medication, and exercise, available for treating hormonal disorders. A number of medicines are available to treat different symptoms associated with PCOS, such as clomiphene citrate, tamoxifen, aromatase inhibitors, metformin, glucocorticoids, or gonadotropins. These pharmacologic strategies, however, have significant limitations including limited efficacy, compliance issue, side effects, and others.

### SUMMARY OF THE INVENTION

One aspect of the invention is directed to a method of treating a hormonal disorder, the method comprises: applying an alternating electric field focused on a liver of the subject at a frequency between approximately 50 kHz and approximately 10,000 kHz, where the subject has polycystic ovary syndrome. In some embodiments, prior to applying an alternating electric field, the subject is not diagnosed as having cancer

One aspect of the invention is directed to a method of a hormonal disorder, the method comprises: increasing a level of Sex Hormone Binding Globulin (SHBG) in blood of the subject by applying an alternating electric field to a liver of the subject at a frequency between approximately 50 kHz and approximately 10,000 kHz. In some embodiments, prior to applying an alternating electric field, the subject is not diagnosed as having cancer.

One aspect of the invention is directed to a method of treating a hormonal disorder in a subject, comprising: applying an alternating electric field to the subject at a frequency between approximately 50 kHz and approximately 10,000 kHz to affect a level of circulating testosterone in the subject. The blood of the subject has a first level of circulating testosterone before applying the alternating electric field, and the blood of the subject has a second level of circulating testosterone after applying the alternating electric field. The second level of circulating testosterone is less than the first level of circulating testosterone. In some embodiments, prior to applying an alternating electric field, the subject is not diagnosed as having cancer.

### BRIEF DESCRIPTION OF THE DRAWINGS

FIG. 1 depicts an example method of treating a hormonal disorder in a subject.

FIG. 2 depicts an example flow chart for the in vitro treatment of a hormonal disorder.

FIGS. 3A-3B depict example effects of alternating electric fields on the secretion of SHBG from ovarian carcinoma (FIG. 3A) and small cell lung carcinoma (FIG. 3B).

FIG. 4 depicts an example graph showing qualitative effects of the alternating electric fields on the secretion of SHBG from ovarian carcinoma and small cell lung carcinoma.

FIG. 5 depicts one example of an apparatus to apply alternating electric fields to a subject's body.

FIG. 6 depicts an example computer apparatus for the control and applications of the alternating electric fields to a subject's body.

### DESCRIPTION OF EMBODIMENTS

An innovative and non-invasive therapeutic approach for the treatment of PCOS and/or hormone disorders is disclosed. Techniques for treating a subject with polycystic ovary syndrome (PCOS) and/or hormonal disorder are disclosed. The present disclosure relates to alternating electric fields, which can be used for decreasing a level of circulating testosterone and/or increasing sex hormone-binding globulin (SHBG) in a body of a subject as a physical modality therapy.

The disclosed techniques can be used for the treatment of a hormonal disorder. The target hormonal disorder can include polycystic ovary syndrome (PCOS). PCOS is a complex endocrinopathy, which can be characterized by menstrual dysfunction, androgen excess, and polycystic ovaries. Certain subjects with PCOS show hyperandrogenism, which can affect the development of the severe endocrine and metabolic disturbances associated with the syndrome.

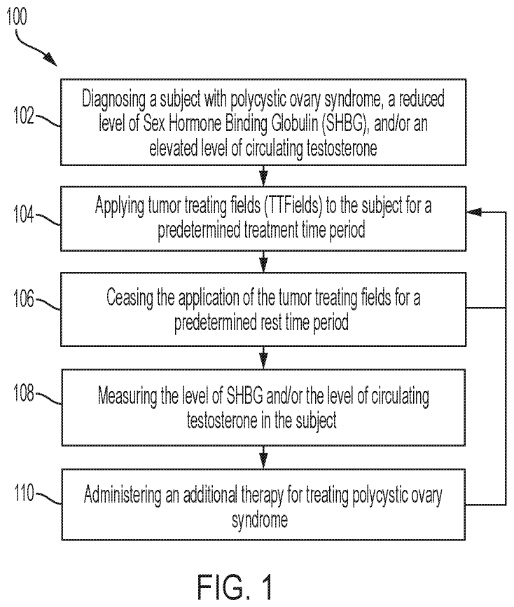

FIG. 1 depicts an example method 100 of treating a hormonal disorder in accordance with the disclosed subject matter. At step 102, the hormonal disorder of the subject can be diagnosed. In one example, the subject can be diagnosed with at least one of PCOS, a reduced level of Sex Hormone Binding Globulin (SHBG), or an elevated level of circulating testosterone. The subject might not be diagnosed as having cancer.

A PCOS diagnosis may be confirmed when certain parameters exist in a subject. These parameters may include, for example, hyperandrogenism, irregular periods (oligomenorrhea), and polycystic ovarian morphology (PCOM) seen on an ultrasound. Hyperandrogenism can be diagnosed based on the presence of excessive acne, androgenic alopecia, hirsutism (terminal hair in a male-pattern distribution), or by elevated serum levels of total, bioavailable, or free testosterone or dehydroepiandrosterone sulfate. A polycystic ovary may be defined as an ovary containing 12 or more follicles (or 25 or more follicles) measuring 2 to 9 mm in diameter, or an ovary with a volume greater than 10 mL as seen on ultrasound. In addition, there are influences of SHBG on altered luteinizing hormone (LH) action and insulin resistance, which may be used to confirm a PCOS diagnosis. Blood tests may be used to identify high androgen levels, such as dehydroepiandrosterone sulphate (DHEAS) or androstenedione.

Various indications and symptoms of PCOS can be treated with the disclosed subject matter. For example, the abnormally low level of testosterone and/or SHBG in the blood of a subject can be the target indication/symptom. SHBG is a protein that is primarily generated by the liver. SHBG is a glycoprotein, which regulates the bioavailability of sex steroid hormones. SHBG can bind to sex hormones in the blood, reducing circulating levels of the free hormones. SHBG concentration can be a major determinant of the metabolic clearance of the sex steroids and their access to target tissues. SHBG's affinity to testosterone is higher than its affinity to estrogen. As a result, most of the circulating testosterone is bound to SHBG. Thus, when SHBG levels drop, levels of free testosterone can rise.

Abnormally low serum SHBG levels can be found in a subject with PCOS and contribute to hyperandrogenic symptoms. A suppressed SHBG level in the presence of a normal level of total testosterone infers an increase in ‘bioavailable’ androgen with a consequent increase in peripheral androgen activity. Furthermore, certain PCOS subjects have elevated testosterone levels. Thus, a low hepatic SHBG production can be a key step in the pathogenesis of PCOS. The disclosed subject matter provides exemplary techniques for increasing the level of SHBG and/or decreasing the level of testosterone for treating a subject with PCOS. In non-limiting embodiments, the disclosed techniques can be used for an early manifestation of PCOS prior to an overt phenotype emerging. Accordingly, the subject with PCOS can be diagnosed by measuring and/or detecting a reduced level of SHBG and/or an elevated level of circulating testosterone from the subject.

The level of circulating SHBG and testosterone can be measured by any methods known in the art such as elisa, competitive electrochemiluminescence immunoassays, or other types of immunoassay. The level of circulating SHBG and testosterone changes due to factors such as sex, age, liver diseases, hyperthyroidism or other metabolic disorders. A normal range of an adult females has a level of circulating SHBG that ranges from 18 nmol/L to 144 nmol/L and a level of circulating that ranges from 15 ng/dL to 70 ng/dL or 0.5 nmodl/L to 2.4 nmodl/L. In some embodiments, the disclosed invention includes immunoassay kits used for the measurement of hormone level in the blood, e.g., the level of circulating SHBG and testosterone.

In one example, the reduced level of SHBG in the subject's blood can be lower than approximately 145 nmol/ml, approximately 125 nmol/ml, approximately 100 nmol/ml, approximately 90 nmol/ml, approximately 80 nmol/ml, approximately 70 nmol/ml, approximately 60 nmol/ml, approximately 50 nmol/ml, approximately 40 nmol/ml, approximately 30 nmol/ml, approximately 20 nmol/ml, or approximately 18 nmol/ml. In one example, the elevated level of testosterone in the subject's blood can be higher than approximately 15 ng/dL, approximately 20 ng/dL, approximately 25 ng/dL, approximately 30 ng/dL, approximately 35 ng/dL, approximately 40 ng/dL, approximately 45 ng/dL, approximately 50 ng/dL, approximately 55 ng/dL, approximately 60 ng/dL, approximately 65 ng/dL, or approximately 70 ng/dL. In one example, the elevated level of testosterone in the subject's blood can be higher than approximately 0.5 nmol/L, approximately 1 nmol/L, approximately 1.5 nmol/L, approximately 2.0 nmol/L, or approximately 2.4 nmol/L.

A “subject” may be a human or a non-human animal, for example, but not by limitation, a non-human primate, a dog, a cat, a horse, a rodent, a cow, a goat, a rabbit, a mouse, etc. The terms “treat,” “treating,” or “treatment,” and other grammatical equivalents as used herein, may include: alleviating, abating, ameliorating, or preventing a disease, condition or symptoms; preventing additional symptoms; ameliorating or preventing the underlying metabolic causes of symptoms; inhibiting the disease or condition (e.g., arresting the development of the disease or condition); relieving the disease or condition; causing regression of the disease or condition; relieving a condition caused by the disease or condition; or stopping the symptoms of the disease or condition. The terms may further include achieving a therapeutic benefit and/or a prophylactic benefit. The terms “simultaneously,” or “concomitantly,” and other grammatical equivalents as used herein, may include applying alternating electric field to a subject while additional therapy or treatment still has an active therapeutic effect in the subject. A therapeutic benefit may include eradication or amelioration of the underlying disorder being treated. Also, a therapeutic benefit may be achieved with the eradication or amelioration of one or more of the physiological symptoms associated with the underlying disorder such that an improvement is observed in the subject, notwithstanding that the subject may still be afflicted with the underlying disorder.

At step 104, an alternating electric field can be applied to the subject for a predetermined treatment time period. The alternating electric field can be focused on a particular area of the subject (e.g., an organ, a tissue, a mass, a part, etc.), for example the liver.

In certain embodiments, transducers with arrays of electrode elements can be placed on a target area of a subject's body, e,g., head, chest, or a surface area that is closest to the target organ, tissue, mass, or cell. The effectiveness of the treatment can be enhanced when the alternating electric field focuses on the target tissue or cells with minimized impact to other tissue or cells.

In certain embodiments, the alternating electric field applied to a subject has an intensity ranging from about 1 v/cm to about 20 v/cm, a frequency from about 50 kHz to about 10,000 kHz, and a duration from about one hour to about 72 hours. The parameters of the treatment depend on the subject's symptoms, severity of the symptoms, age, size, metabolism, and/or whether additional treatment therapy is administered. As an example, an older subject with more severe symptoms, bigger body size, and lower metabolism can require greater treatment intensity, frequency, and longer treatment time. On the contrary, lower treatment intensity, frequency, and shorter treatment time may be applied when additional treatment therapy is adopted.

In one example, the alternating electric field can be a low intensity alternating electric field within the intermediate frequency range. For example, an alternating electric field is induced non-invasively into a region of interest by transducers placed directly on the subject's body and applying AC voltages between the transducers. In one example, the alternating electric field can include tumor treating fields. In one example, the alternating electric field is not a tumor treating field and targets non-cancer/tumor tissue. For example, the target tissue of the disclosed subject matter can be the liver of the subject, and the subject does not have cancer (or has not been diagnosed with cancer). The alternating electric field can alternate approximately 100,000 to approximately 1,000,000 times per second, for example, 100,000 to approximately 300,000 times per second, while it is applied to a target tissue/cell (e.g., liver) of a subject.

In non-limiting embodiments, the alternating electric field can be applied with predetermined parameters. As an example, the alternating electric field can include a frequency within a frequency range from about 50 kHz to about 10 MHz, from about 50 kHz to about 5 MHz, or from about 50 kHz to about 1000 kHz. As an example, the frequency of the alternating electric field may be between approximately 100 kHz and approximately 500 kHz. As an example, the frequency of the alternating electric field may be between approximately 1 MHz and approximately 10 MHz. As an example, the frequency of the alternating electric field may be approximately 100 kHz, approximately 150 kHz, approximately 200 kHz, or approximately 250 kHz. The frequency may be between approximately 150 kHz and approximately 200 kHz. As an example, the frequency of the alternating electric field may be approximately 1 MHz, approximately 2 MHz, approximately 3 MHz, approximately 4 MHz, approximately 5 MHz, approximately 6 MHz, approximately 7 MHz, approximately 8 MHz, approximately 9 MHz, or approximately 10 MHz.

As an example, the alternating electric field may include an intensity within an intensity range from about 1 V/cm to about 20 V/cm. As an example, the alternating electric field may include an intensity within an intensity range from about 1 V/cm to about 10 V/cm. As an example, the intensity of the alternating electric field may be between approximately 1 V/cm and approximately 4 V/cm.

Other possible exemplary parameters for the alternating electric field may include active time, dimming time, and duty cycle (all of which may be measured in, for example, ms units), among other parameters. The parameters can be modified based on the sizes of the target tissue (e.g., liver), subjects, or purposes of the treatment. In one example, the intensity of the tumor treating field alternating electric field is between approximately 1 V/cm and approximately 4 V/cm, and the frequency of the alternating electric field is between approximately 150 kHz and approximately 250 kHz for treating a subject with PCOS.

In one example, the predetermined treatment time period can be between approximately one hour to approximately 72 hours. For example, the predetermined treatment time can be approximately one hour, approximately two hours, approximately three hours, approximately four hours, approximately five hours, approximately ten hours, approximately 12 hours, approximately 24 hours, approximately 48 hours, or approximately 72 hours. The predetermined treatment time period can be modified based on the subject (e.g., age or size) and/or severity of the hormonal disorder.

In certain embodiments, when the alternating electric field is applied, the subject has a diagnosis of at least one of PCOS, a reduced level of Sex Hormone Binding Globulin (SHBG), or an elevated level of circulating testosterone. In certain embodiments, when the alternating electric field is applied, the subject does not have a diagnosis of cancer.

At step 106, the application of the alternating electric field can be ceased for a rest time period. During the rest time period, transducer the arrays may remain in place on the subject's body while the alternating electric fields are not applied. In another example, the transducer arrays can be removed during the predetermined rest time period, and then replaced with new transducer arrays (or the same transducer arrays) at the conclusion of the rest time period. The duration of the rest time period may be predetermined by the treatment protocol and/or by the subject. The rest time period can be from approximately ten seconds to approximately a week. For example, the rest time period can be approximately ten seconds, approximately a minute, approximately ten minutes, approximately 30 minutes, approximately one hour, approximately two hours, approximately three hours, approximately four hours, approximately five hours, approximately ten hours, approximately 12 hours, approximately 24 hours, approximately 48 hours, or approximately 72 hours. The rest time period can be modified based on the subject (e.g., age or size), severity of the hormonal disorder, and/or whether additional therapy is applied.

In one example, the alternating electric field can be administered to the subject by cycling between the predetermined treatment time period when the alternating electric field is applied to the subject and a predetermined rest time period when the alternating electric field is not applied to the subject. In non-limiting embodiments, the number of cycles can be from approximately 1 to approximately 50, or from approximately 1 to approximately 24.

In one example, the treatment may include pretermined treatment time periods and short rest time periods during which the alternatic electric fields are not being applied to the subject. For example, during a 24 hour (or greater) treatment time period, the alternating electric field may be applied for at least 80% of the treatment time period. For example, during a 24 hour treatment time period, the alternating electric field may be applied for at least 19 hours of the treatment time period. The rest time periods may be predetermined according to the treatment protocol and/or may be determined by the subject.

In another example, alternating electric fields may be applied to the subject without a rest time period.

In non-limiting embodiments, the cycling treatment between treatment time periods and rest time periods may stop when the level of SHBG in the subject's blood is increased to the target level or the level of testosterone in the subject's blood is decreased to the target level.

At step 108, the level of SHBG and/or the level of circulating testosterone in the subject can be measured. For example, the level of SHBG and/or the level of circulating testosterone can be measured after applying the alternating electric field to the subject to confirm whether the level of SHBG in the subject's blood is increased to the target level or the level of testosterone in the subject's blood is decreased to the target level. In one example, the target level of the SHBG can be between approximately 18 nmol/L to approximately 144 nmol/L after applying the alternating electric field to a target tissue/cell (e.g., liver) of the subject. In one example, the target level of the circulating testosterone can be between approximately 15 ng/dL to approximately 70 ng/dL after applying the alternating electric field to a target tissue/cell (e.g., liver) of the subject. In one example, the target level of the circulating testosterone can be between approximately 0.5 nmol/L to approximately 2.4 nmol/L after applying the alternating electric field to a target tissue/cell (e.g., liver) of the subject. In one example, the subject is diagnosed as having no PCOS or reduced PCOS after applying the alternating electric field to a target tissue/cell (e.g., liver) of the subject.

At step 110, additional therapy can be administered to treat a hormonal disorder of a subject. In one example, the additional therapy can be a therapy for treating PCOS. The additional therapy may include, for example, surgery, a medicine, or a combination thereof. In one example, the additional therapy can include the administration of metformin (MET), tamoxifen, clomiphene citrate, glucocorticoids, gonadotropins, aromatase inhibitors, diabetes treatment (for example, a glucagon-like peptide-1 receptor agonist (GLP-1 RA)), a thiazolidinedione (TZD), a compound oral contraceptive (COCs), or a combination thereof.

In certain embodiments, the additional therapy can be delivered to the subject through various techniques. In one example, the additional therapy can be administered to the subject orally. For example, the disclosed active agents (e.g., MET, GLP-1 RA, TZD, and COCs) can be formulated as tablets, pills, capsules, liquids, gels, syrups, slurries, suspensions and the like, for oral or nasal ingestion by a subject to be treated. In certain embodiments, the disclosed active agents can be an immediate-release tablet. Alternatively or additionally, the disclosed active agents can be extended or controlled-release tablets. In one example, the disclosed inhibitors can be administered to the subject parenterally. The terms “parenteral administration” and “administered parenterally,” as used herein, refers to the administration other than enteral and topical administration, usually by injection, and includes, without limitation, intravenous, intramuscular, intraarterial, intrathecal, intracapsular, intracardiac, intradermal, intraperitoneal, transtracheal, subcutaneous, subcuticular, intraarticular, subcapsular, subarachnoid, intraspinal, epidural and intrasternal injection and infusion. For example, and not by way of limitation, the disclosed active agent can be administered to a subject intravenously. The disclosed active agent can also be administered intravaginally.

In certain embodiments, the alternating electric field can be applied to a target tissue/cell (e.g., liver) of the subject before or after the additional therapy is administered. In some instances, the alternating electric field is applied to a target area, organ, tissue, or cell of the subject both before and after the additional therapy is administered. In certain embodiments, the alternating electric field can be simultaneously applied to the target tissue with the disclosed additional therapy. As an example, at least a portion of the applying step 104 may be performed simultaneously/concomitantly with at least a portion of the administration step 110.

In some embodiments, an alternating electric field is applied to the target tissue of a subject right before or after the administration of additional therapy, e.g. a MET, a TZD, a GLP-1, a COC, a tamoxifen, a tamoxifen, a clomiphene citrate, a glucocorticoid, a gonadotropin, an aromatase inhibitor or a combination thereof, to increase the subject's response to the additional therapy. As an example, an alternating electric field is applied to the target tissue of a subject right before or after the administration of a MET, a TZD, a GLP-1, a COC, a tamoxifen, a tamoxifen, a clomiphene citrate, a glucocorticoid, a gonadotropin, or an aromatase inhibitor, and a subsequential administration of the same or a different drug selected from the group consisting of a MET, a TZD, a GLP-1, a COC, a tamoxifen, a tamoxifen, a clomiphene citrate, a glucocorticoid, a gonadotropin, and an aromatase inhibitor. In some embodiments, an alternating electric field is applied to the target tissue of a subject some time, e.g., 1 hour, 1 week, or 1 month, before or after the administration of additional therapy, wherein the administration mode of the additional therapy, e.g. drug dosage, frequency, duration, route of administration, can be adjusted based on the therapeutic effects of the alternating electric field. In some embodiments, a subject receives multiple alternations between the alternating electric field treatment and the administration of additional therapy to achieve a better clinical outcome depending on the subject's response to the alternating electric field treatment and the additional therapy. In certain embodiments, a subject receives the administration of additional therapy during 5%, 10%, 20%, 30%, 40%, 50%, 60%, 70%, 80%, 90%, or 100% of the whole time period while an alternating electric field is applied. The time period of the administration of additional therapy is defined as the duration of which the medicine has an active therapeutic effect for the treatment of hormone disorder or PCOS.

In some instances, an alternating electric field is applied to the target tissue of a subject right before or after the administration of a MET, a TZD, a GLP-1, a COC, a tamoxifen, a tamoxifen, a clomiphene citrate, a glucocorticoid, a gonadotropin, or an aromatase inhibitor; a subsequent alternating electric field is applied to the target tissue of the subject right before or after the administration of the same or a different therapeutic drug selected from the group consisting of a MET, a TZD, a GLP-1, a COC, a tamoxifen, a tamoxifen, a clomiphene citrate, a glucocorticoid, a gonadotropin, and an aromatase inhibitor; wherein a rest time period between the first and the subsequent alternating electric field treatments ranges between 0 to 1 week. In some embodiments, multiple subsequent alternating electric field treatments may be applied.

In some instances, an alternating electric field is applied to the target tissue of a subject while the administration of a MET, a TZD, a GLP-1, a COC, a tamoxifen, a tamoxifen, a clomiphene citrate, a glucocorticoid, a gonadotropin, or an aromatase inhibitor; a subsequent alternating electric field is applied to the target tissue of the subject while the administration of the same or a different therapeutic drug selected from the group consisting of a MET, a TZD, a GLP-1, a COC, a tamoxifen, a tamoxifen, a clomiphene citrate, a glucocorticoid, a gonadotropin, and an aromatase inhibitor; wherein a rest time period between the first and the subsequent alternating electric field treatments ranges between 0 to 1 week. In some embodiments, multiple subsequent alternating electric field treatments may be applied.

In certain embodiments, the application of alternating electric field treatment together with the administration of additional therapy has synergistic effects in increasing the circulating SHBG to the target level, decreasing the circulating testosterone to the target level, and/or reducing or eliminating the symptoms of PCOS. As an example, the alternating electric field treatment can shorten the treatment period of a subject undergone additional therapy or reduce the dose of additional pharmaceutical therapy to achieve the same or better therapeutic effects.

In certain embodiments, the disclosed techniques can be used for increasing SHBG and decreasing the circulating testosterone in blood in a subject. For example, the increased SHBG can increase the level of SHBG-testosterone complex resulting in the decreased circulating testosterone in the blood of a subject. The blood of the subject can have a first level of circulating testosterone before applying the alternating electric field and a second level of circulating testosterone after applying the alternating electric field. The first and second levels can be measured using any method known in the art before and after applying the alternating electric field. The second level of circulating testosterone is less than the first level of circulating testosterone after applying the alternating electric field.

In one example, the target tissue/cell of the alternating electric field can be a liver, an ovary, a brain, a liver cell, an ovarian cell, a brain cell, or a combination thereof. The alternating electric field can be focused on the target tissue/cell, and the alternating electric field is not applied to the entire subject.

The dosage and administration plan can vary depending on the subject (e.g., based on the subject's age, metabolism, symptoms, ovulation/menstruation cycle, and so forth) and the target tissue. For example, to reduce or eliminate symptoms of PCOS and oligomenorrhea, alternating electric fields may be applied to the liver of a subject for about one to ten consecutive days, e.g. about 1 day, about 2 days, about 3 days, about 4 days, about 5 days, about 7 days, about 10 days, or at least 24 hours every ovulation/menstruation cycle of the subject, e.g. every 24 days to every 32 days. More particularly, the alternating electric fields may be applied to the liver for 72 consecutive hours every 24 days to every 32 days, e.g., 28 days. In some embodiments, an alternating electric fields may be applied to the liver of a subject periodically for about 12 days to about the whole period of the subject's each ovulation/menstruation cycle, such that the accumulative time of alternating electric fields treatment is greater than 50%, 60%, 70%, 80%, and 90% of the subject's every ovulation/menstruation cycle. More particularly, in some instances, the alternating electric fields may be applied to the liver of the subject every other day during the subject's each ovulation/menstruation cycle, e.g. about 15 days alternating electric fields treatment every 29 days. In some other instances, the alternating electric fields may be applied to the liver of the subject for a consecutive 48 hours, 72 hours, 96 hours, or 120 hours, followed by a rest period, e.g. a 6-hour rest, a 12-hour rest, or a 24-hour rest. The same or different treatment-rest patterns repeat and continue from the beginning to the end of the subject's each ovulation/menstruation cycle. Such an application of alternating electric fields may increase estrogen levels in the subject's body, or estrogen efficacy due to the testosterone being bound to SHBG, thereby providing the physiological increase in estrogen levels that normally occurs during the time period preceding ovulation each month. In another example, alternating electric fields may be applied for at least 24 hours every 7 days to continuously provide an increase in SHBG levels in the subject's body over time.

### Experimental Results

Sex hormone-binding globulin (SHBG) is a protein that is primarily generated by the liver. SHBG is a glycoprotein, which regulates the bioavailability of sex steroid hormones. SHBG can bind to sex hormones in the blood, reducing circulating levels of the free hormones. SHBG concentration is a major determinant of the metabolic clearance of the sex steroids and their access to target tissues. SHBG's affinity to testosterone is higher than its affinity to estrogen. As a result, most of the circulating testosterone is bound to SHBG. Thus, when SHBG levels drop, levels of free testosterone can rise.

Polycystic ovary syndrome (PCOS) is a highly prevalent but complex endocrinopathy characterized by menstrual dysfunction, androgen excess, and polycystic ovaries. Between 70-80% of PCOS women show hyperandrogenism, which can play a major pathological role in the development of the severe endocrine and metabolic disturbances associated with the syndrome.

Abnormally low serum SHBG levels are frequently found in women with PCOS and contribute to hyperandrogenic symptoms. Total testosterone and SHBG concentrations are interdependent. A suppressed SHBG in the presence of normal total testosterone infers an increase in ‘bioavailable’ androgen with a consequent increase in peripheral androgen activity. PCOS subjects often have elevated testosterone levels. Thus, a low hepatic SHBG production can be a key contributor to the pathogenesis of PCOS, and the SHBG reduction can be an early manifestation of PCOS prior to an overt phenotype emerging.

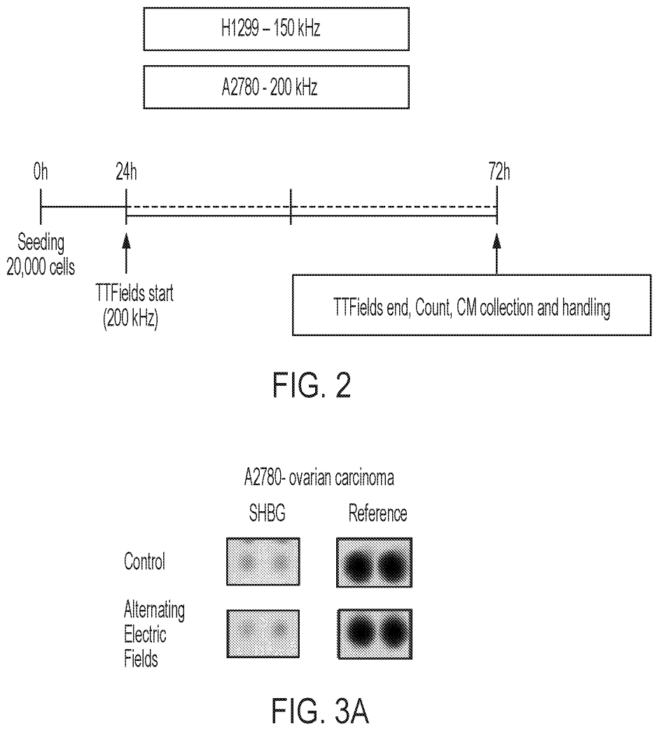

Using certain embodiments disclosed herein, alternating electric fields were applied to increase the level of secreted SHBG and to treat hormonal disorders (e.g., PCOS). For example, as shown in FIG. 2, about 20,000 ovarian carcinoma cells or small cell lung carcinoma cells were seeded on culture dishes. After 24 hours, the cells were exposed to alternating electric fields (e.g., 150 kHz for the small cell lung carcinoma cells and 200 kHz for the ovarian carcinoma) for 72 hours. After the alternating electric field treatments, the cell numbers were counted, cell media was collected as conditioned media and cells were harvested for further analysis (e.g., gene, protein expression, and/or secreted cytokine analyses). For example, for the cytokine array analysis of proteins secreted from ovarian cancer cell line A2780 or non-small cell lung carcinoma H1299, volumes of conditioned media were evened using molecular grade distilled water. The samples were centrifuged, and upper fluid was collected and kept at −20 degrees. The level of SHBG in the conditioned media was determined using Proteome Profiler Human XL Cytokine Assay kit (IRA022B) according to its user manual. SHBG concentration is proportional to the pixel density from the cytokine array membrane and can be quatified using a transmission-mode scanner and image analysis software. Cell number was normalized after Flow cytometer (FACS) count.

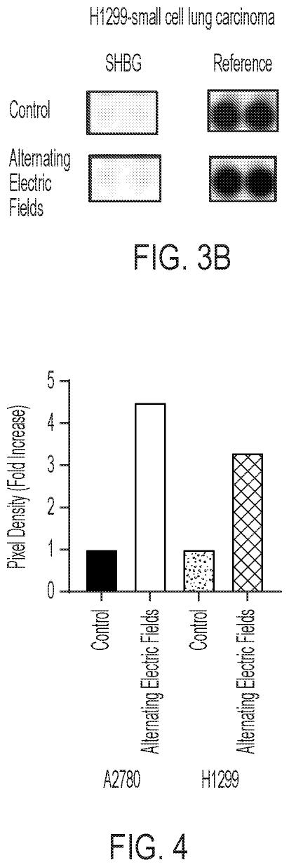

As shown in FIGS. 3A, 3B, and 4, ovarian cancer cell line A2780 and non-small cell lung carcinoma H1299 showed the increased SHBG levels after application of an alternating electric field. The fold change of more than 3 was considered as up-regulation following the alternating electric fields treatment. The qualitative results of FIG. 4 show that after alternating electric field application for 72 hours, ovarian cancer cell line A2780 and non-small cell lung carcinoma H1299 showed the secreted SHBG levels increased by 4.5 and 3.3 fold, respectively, in condition media derived from cell culture.

The genes, proteins, and/or secreted cytokine revealed and alterned in response to the alternating electric field treatment disclosed herein can serve as additional biomarks or targets for the diagonosis and treatment of the PCOS and/or associated-hormonal disorders.

### Exemplary Apparatuses

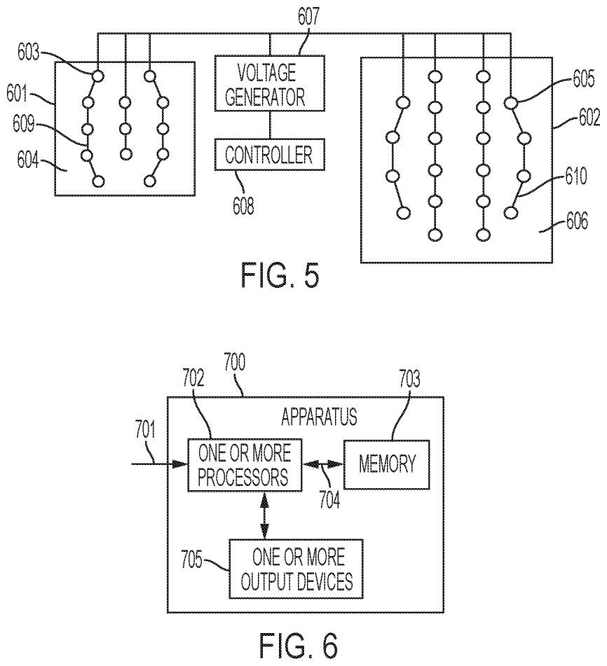

FIG. 5 depicts one example of an apparatus to apply an alternating electric field to a subject's body. The first array 601 includes electrode elements 603, which are positioned on the substrate 604, and the electrode elements 603 are electrically and mechanically connected to one another through a conductive wiring 609. The second array 602 includes electrode elements 605, which are positioned on the substrate 606, and the electrode elements 605 are electrically and mechanically connected to one another through a conductive wiring 610. As illustrated, the first and second arrays 601 and 602 may include different numbers of electrode elements 603 and 605, respectively. In other examples, the first and second arrays 601 and 602 may each have the same number of electrode elements 603 and 605 thereon. The first array 601 and the second array 602 are connected to an AC voltage generator 607 and a controller 608. The controller 608 may include one or more processors and memory accessible by the one or more processors. The memory may store instructions that when executed by the one or more processors, control the AC voltage generator 607 to implement one or more embodiments of the invention. In some embodiments, the AC voltage generator 607 and the controller 608 may be integrated in the first array 601 and the second array 602 and form a first electric field generator and a second electric field generator.

In an exemplary embodiment, in applying an alternating electric field to a subject, the subject may have four arrays placed on the subject, and the alternating electric field may be focused on a particular organ, tissue, cells, or area of the subject (e.g., the liver). The four transducers may have the same size or one or more different sizes. The four transducers may have the same layout and/or number of electrode elements or one or more different layouts and/or numbers of electrode elements. In applying an alternating electric field to a subject, the electric field may alternate between a first pair of arrays placed on the subject and a second pair of arrays placed on the subject. To focus an alternating electric field on a particular area of a subject, a first line between the first pair of arrays and a second line between the second pair of arrays may intersect at the particular area of the subject, and computer-based simulations and calculations can be performed to determine one or more optimal locations to place the arrays on the subject.

In another exemplary embodiment, in applying an alternating electric field to a subject, the subject may have two arrays placed on the subject, and the alternating electric field may be focused on a particular organ, tissue, cells, or area of the subject (e.g., the liver). The two transducers may have the same size or one or more different sizes. The two transducers may have the same layout and/or number of electrode elements or one or more different layouts and/or numbers of electrode elements. In applying an alternating electric field to a subject, the alternating electric field may be generated between a pair of arrays placed on the subject. To focus an alternating electric field on a particular area of a subject, a line between the pair of arrays may pass through the particular area of the subject, and computer-based simulations and calculations can be performed to determine one or more optimal locations to place the arrays on the subject.

FIG. 6 depicts an example computer apparatus for use with the embodiments herein. As an example, the apparatus 700 may be a computer to implement certain inventive techniques disclosed herein. As an example, the apparatus 700 may be a controller apparatus to apply alternating electric fields for the embodiments herein. The controller apparatus 700 may be used as the controller 608 of FIG. 5. The apparatus 700 may include one or more processors 702, one or more output devices 705, and a memory 703.

In one example, based on input 701, the one or more processors generate control signals to control the voltage generator to implement an embodiment of the invention. In one example, the input 701 is user input. In another example, the input 701 may be from another computer in communication with the controller apparatus 700. The output devices 705 may provide the status of the operation of the invention, such as transducer selection, voltages being generated, and other operational information. The output devices 705 may provide visualization data according to certain embodiments of the invention.

The memory 703 may be accessible by the one or more processors 702 via the link 704 so that the one or more processors 702 can read information from and write information to the memory 703. The memory 703 may store instructions that when executed by the one or more processors 702 implement one or more embodiments of the invention.

### ILLUSTRATIVE EMBODIMENTS

The invention includes other illustrative embodiments, such as the following.

Illustrative Embodiment 1. A method of treating a subject, comprising: applying an alternating electric field focused on a liver of the subject at a frequency between approximately 50 kHz and approximately 10 MHz, wherein the subject has polycystic ovary syndrome.

Illustrative Embodiment 2. The method of Illustrative Embodiment 1, wherein the frequency is between approximately 50 kHz and approximately 1 MHz, wherein the subject has polycystic ovary syndrome.

Illustrative Embodiment 3. The method of any one of Illustrative Embodiments 1 and 2, wherein the frequency is between approximately 150 kHZ and approximately 200 kHZ.

Illustrative Embodiment 4. The method of any one of Illustrative Embodiments 1-3, wherein the alternating electric field is administered to the subject by cycling between a predetermined treatment time period when the alternating electric field is applied to the subject and a predetermined rest time period when the alternating electric field is not applied to the subject.

Illustrative Embodiment 5. The method of Illustrative Embodiment 4, wherein the predetermined treatment time period is between approximately one hour to approximately 72 hours.

Illustrative Embodiment 6. The method of any one of Illustrative Embodiments 4-5, wherein the predetermined rest time period is approximately a week.

Illustrative Embodiment 7. The method of any one of Illustrative Embodiments 1-6, further comprises administering an additional therapy for treating the polycystic ovary syndrome, wherein the additional therapy comprises a surgery and/or an administration of a medicine selected from the group consisting of a metformin (MET), a tamoxifen, a clomiphene citrate, a glucocorticoid, a gonadotropin, an aromatase inhibitor, a glucagon-like peptide-1 receptor agonist (GLP-1 RA), a thiazolidinedione (TZD), a compound oral contraceptive (COCs), and a combination thereof.

Illustrative Embodiment 8. The method of any one of Illustrative Embodiments 1-7, further comprises applying an alternating electric field to a target area, organ, tissue, mass, or cell of the subject before and/or after the additional therapy is administered; or applying at least a portion of an alternating electric field to a target area, organ, tissue, mass, or cell of the subject simultaneously, before, or after at least a portion of the additional therapy is administered.

Illustrative Embodiment 9. The method of any one of Illustrative Embodiments 1-8, further comprises diagnosing a subject with at least one of polycystic ovary syndrome, a reduced level of Sex Hormone Binding Globulin (SHBG), or an elevated level of circulating testosterone.

Illustrative Embodiment 10. The method of any one of Illustrative Embodiments 1-9, further comprises applying an alternating electric field focused on a target region, organ, tissue, mass, or cell of the subject, wherein the alternating electric field is programed to cycle on and off for a predetermined period of time or for multiple periods of time; wherein the number of cycles ranges from approximately 1 to approximately 50; wherein the alternating electric field when applied to the subject has an intensity from about 1 v/cm to about 20 v/cm, a frequency from about 50 kHz to about 10,000 kHz, and a duration from about one hour to about 72 hours; wherein the rest time period when the alternating electric field is not applied to the subject ranges from 0 to a week; wherein the subject has polycystic ovary syndrome, a hormonal disorder, or a combination thereof.

Illustrative Embodiment 11. The method of any one of Illustrative Embodiments 1-10, further comprises determining the level of SHBG in a sample from the subject to which the alternating electric field applied, wherein the level of SHBG increases after the alternating electric field treatment; determining the level of testosterone in a sample from the subject to which the alternating electric field applied, wherein the level of testosterone decreases after the alternating electric field treatment; and/or measuring polycystic ovary syndrome parameter of the subject to which the alternating electric field applied, wherein the polycystic ovary syndrome is improved or eliminated after the alternating electric field treatment.

Illustrative Embodiment 12, The method of any one of Illustrative Embodiments 1-11, wherein the subject is not diagnosed as having a cancer.

Illustrative Embodiment 13. A method of treating a subject, comprising: increasing a level of Sex Hormone Binding Globulin (SHBG) in blood of the subject by applying the alternating electric field focused on the liver of the subject.

Illustrative Embodiment 14, The method of Illustrative Embodiment 13, wherein the frequency is between approximately 50 kHz and approximately 10 MHz.

Illustrative Embodiment 15. The method of Illustrative Embodiment 13, wherein the frequency is between approximately 50 kHz and approximately 1 MHz.

Illustrative Embodiment 16. The method of any one of Illustrative Embodiments 1 and 2, wherein the frequency is between approximately 150 kHz and approximately 200 kHz.

Illustrative Embodiment 17. The method of any one of Illustrative Embodiments 13-16, wherein the level of SHBG in the blood of the subject is less than approximately 144 nmol/L before applying the alternating electric field to the liver of the subject.

Illustrative Embodiment 18. The method of any one of Illustrative Embodiments 13-17, further comprises measuring the level of SHBG in the blood of the subject after applying the alternating electric field to the liver of the subject.

Illustrative Embodiment 19. The method of any one of Illustrative Embodiments 13-18, wherein the level of SHBG in the blood of the subject is between approximately 18 nmol/L to approximately 144 nmol/L after applying the alternating electric field to the liver of the subject.

Illustrative Embodiment 20. The method of any one of Illustrative Embodiments 13-19, wherein the alternating electric field is administered to the subject by cycling between a predetermined treatment time period when the alternating electric field applied to the subject and a predetermined rest time period when the tumor treating fields are not applied to the subject.

Illustrative Embodiment 21. The method of any one of Illustrative Embodiments 13-20, wherein a level of SHBG-testosterone complex is increased in the blood of the subject after applying the alternating electric field.

Illustrative Embodiment 22. The method of any one of Illustrative Embodiments 1-12, wherein when the alternating electric field is applied, the subject has a diagnosis of at least one of polycystic ovary syndrome, a reduced level of Sex Hormone Binding Globulin (SHBG), or an elevated level of circulating testosterone.

Illustrative Embodiment 23. The method of any one of Illustrative Embodiments 13-21, further comprising diagnosing the subject with at least one of polycystic ovary syndrome, a reduced level of Sex Hormone Binding Globulin (SHBG), or an elevated level of circulating testosterone.

Illustrative Embodiment 24. The method of any one of Illustrative Embodiments 1-12, wherein, when the alternating electric field is applied, the subject does not have a diagnosis of cancer

Illustrative Embodiment 25. The method of Illustrative Embodiment 13-21, wherein the subject is not diagnosed as having a cancer.

Illustrative Embodiment 26. A method of treating a subject, comprising: applying an alternating electric field to the subject at a frequency between approximately 50 kHz and approximately 10,000 kHz to affect a level of circulating testosterone in the subject, wherein blood of the subject has a first level of circulating testosterone before applying the alternating electric field, wherein blood of the subject has a second level of circulating testosterone after applying the alternating electric field, wherein the second level of circulating testosterone is less than the first level of circulating testosterone.

Illustrative Embodiment 27. The method of Illustrative Embodiment 26, wherein the frequency is between approximately 150 kHZ and approximately 200 kHZ.

The method of Illustrative Embodiment 28. The method of any one of Illustrative Embodiments 26 and 27, wherein the first level of the circulating testosterone is greater than or equal to approximately 70 ng/dL or greater than or equal to approximately 2.4 nmol/L.

Illustrative Embodiment 29. The method of any one of Illustrative Embodiments 26-28, wherein the second level of the circulating testosterone is less than or equal to approximately 70 ng/dL or less than or equal to approximately 2.4 nmol/L.

Illustrative Embodiment 30. The method of any one of Illustrative Embodiments 26-29, further comprises measuring the level of the circulating testosterone in the blood of the subject before and after applying the alternating electric field to the subject.

Illustrative Embodiment 31. The method of any of Illustrative Embodiments 26-30, wherein the alternating electric field is administered to the subject by cycling between a predetermined treatment time period when the alternating electric field is applied to the subject and a predetermined rest time period when the alternating electric field is not applied to the subject.

Illustrative Embodiment 32. The method of any one of Illustrative Embodiments 26-31, wherein the alternating electric field is applied to at least one of a liver cell, an ovarian cell, or a brain cell.

Illustrative Embodiment 33. The method of any one of Illustrative Embodiments 26-32, further comprises diagnosing a subject with at least one of polycystic ovary syndrome, a reduced level of Sex Hormone Binding Globulin (SHBG), or an elevated level of circulating testosterone.

Illustrative Embodiment 34. The method of any one of Illustrative Embodiments 26-33, wherein, when the alternating electric field is applied, the subject does not have a diagnosis of cancer.

Illustrative Embodiment 35. An apparatus for the treatment of a polycystic ovary syndrome, a hormonal disorder, or a combination thereof of a subject, the apparatus comprising: an voltage generator; a controller, wherein the controller may include one or more processors and memory accessible by one or more processors that control the voltage generator; a set of at least four insulated transducers with arrays of electrode elements, wherein the electrode elements are electrically and mechanically connected to one another through a conductive wiring, wherein each array and its electrode elements are positions on a substrate to form the transducer that may be placed against the subject's body, organ, tissue, mass, or cells; wherein the voltage generator, the controller, and the transducers are configured so that, when the transducers are placed against the target's body, organ, tissue, mass, or cells, an alternating electric field is imposed in the target region of the subject to alleviate the polycystic ovary syndrome, increase the level of circulating SHBG, and/or decrease the level of circulating testosterone.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments but that it has the full scope defined by the language of the following claims and equivalents thereof.

What is claimed is:

1. A method of treating a subject, comprising:
applying an alternating electric field focused on a liver of the subject at a frequency between approximately 50 kHz and approximately 10,000 kHz, wherein the subject has polycystic ovary syndrome.

2. The method of claim 1, wherein the frequency is between approximately 150 kHZ and approximately 200 kHZ.

3. The method of claim 1, wherein the alternating electric field is administered to the subject by cycling between a predetermined treatment time period when the alternating electric field is applied to the subject and a predetermined rest time period when the alternating electric field is not applied to the subject.

4. The method of claim 3, wherein the predetermined treatment time period is between approximately one hour to approximately 72 hours,
wherein the predetermined rest time period is approximately a week.

5. The method of claim 1, further comprising administering an additional therapy for treating the polycystic ovary syndrome, wherein the additional therapy comprises an administration of a metformin (MET), a glucagon-like peptide-1 receptor agonist (GLP-1 RA), a thiazolidinedione (TZD), a compound oral contraceptive (COCs), or a combination thereof.

6. The method of claim 1, further comprising diagnosing a subject with at least one of polycystic ovary syndrome, wherein the additional therapy comprises a surgery, and/or an administration of a medicine selected from the group consisting of a metformin (MET), a tamoxifen, a clomiphene citrate, a glucocorticoid, a gonadotropin, an aromatase inhibitor, a glucagon-like peptide-1 receptor agonist (GLP-1 RA), a thiazolidinedione (TZD), a compound oral contraceptive (COCs), and a combination thereof.

7. The method of claim 6, wherein the subject is not diagnosed as having a cancer.

8. A method of treating a subject, comprising:
increasing a level of Sex Hormone Binding Globulin (SHBG) in blood of the subject by applying the alternating electric field focused on the liver of the subject.

9. The method of claim 8, wherein the level of SHBG in the blood of the subject is less than approximately 144 nmol/L before applying the alternating electric field to the liver of the subject.

10. The method of claim 8, further comprising measuring the level of SHBG in the blood of the subject after applying the alternating electric field to the liver of the subject.

11. The method of claim 8, wherein the level of SHBG in the blood of the subject is between approximately 18 nmol/L to approximately 144 nmol/L after applying the alternating electric field to the liver of the subject.

12. The method of claim 8, wherein a level of SHBG-testosterone complex is increased in the blood of the subject after applying the alternating electric field.

13. The method of claim 8, wherein, when the alternating electric field is applied, the subject has a diagnosis of at least one of polycystic ovary syndrome, a reduced level of Sex Hormone Binding Globulin (SHBG), or an elevated level of circulating testosterone.

14. The method of claim 13, wherein, when the alternating electric field is applied, the subject does not have a diagnosis of cancer.

15. A method of treating a subject, comprising:
applying an alternating electric field to the subject at a frequency between approximately 50 kHz and approximately 10,000 kHz to affect a level of circulating testosterone in the subject,
wherein blood of the subject has a first level of circulating testosterone before applying the alternating electric field,
wherein blood of the subject has a second level of circulating testosterone after applying the alternating electric field,
wherein the second level of circulating testosterone is less than the first level of circulating testosterone.

16. The method of claim 15, wherein the first level of the circulating testosterone is greater than or equal to approximately 70 ng/dL or greater than or equal to approximately 2.4 nmol/L.

17. The method of claim 15, wherein the second level of the circulating testosterone is less than or equal to approximately 70 ng/dL or less than or equal to approximately 2.4 nmol/L.

18. The method of claim 15, further comprising measuring the level of the circulating testosterone in the blood of the subject before and after applying the alternating electric field to the subject.

19. The method of claim 15, wherein the alternating electric field is focused on at least one of a liver cell, an ovarian cell, or a brain cell.

20. The method of claim 15, further comprising diagnosing a subject with polycystic ovary syndrome, a reduced level of Sex Hormone Binding Globulin (SHBG), and/or an elevated level of circulating testosterone.
